(19) United States
(12) Patent Application Publication
(10) Pub. No.: US 2003/0007988 A1
(43) Pub. Date: Jan. 9, 2003

(54) Slimming cosmetic composition comprising a plant extract containing a plant natriuretic peptide (PNP)

(75) Inventor: Olivier Courtin, Boulogne sur Seine (FR)

Correspondence Address: SCHNADER HARRISON SEGAL & LEWIS, LLP, 1600 MARKET STREET, SUITE 3600, PHILADELPHIA, PA, 19103

(73) Assignee: Laboratoires Clarins

(21) Appl. No.: 10/198,293

(22) Filed: Jul. 18, 2002

Related U.S. Application Data
(63) Continuation of application No. PCT/FR01/00160, filed on Jan. 18, 2001.

(30) Foreign Application Priority Data
Jan. 28, 2000 (FR) FR 00/01151

(51) Int. Cl. A61K 35/78; A61K 31/522; A61K 7/00

(52) U.S. Cl. 424/401; 424/725; 424/770; 424/754; 514/263.31; 424/769

(57) ABSTRACT

The invention concerns a slimming cosmetic composition characterized in that it includes PNP as active agent and, more particularly, a plant extract containing PNP.

### RELATED APPLICATION

This is a continuation of International Application No. PCT/FR01/00160, with an international filing date of Jan. 18, 2001, which is based on French Patent Application No. FR 00/01151, filed Jan. 28, 2000.

### FIELD OF THE INVENTION

This invention relates to the use of ANP and more specifically an extract of plant origin containing ANP, hereafter designated “PNP” or “plant natriuretic peptide” to promote lipolysis, which is useful for preparing slimming cosmetic products.

### BACKGROUND

The hypodermis or adipose tissue forms a layer of reserve fat which completely envelopes the body. However, its thickness is variable. In men, it is prominent above the waist, principally in the abdomen. In women, it is concentrated below the waist, in the zone of the buttocks, thighs and lower part of the abdomen. The adipose tissue represents 15 to 20% of the body weight, which corresponds to 50 billion adipose cells or adipocytes. The adipocyte is capable of storing lipids in the form of triglycerides (TG) or conversely to mobilize rapidly triglycerides that are the major energy sources of the organism. Regulation of these two processes depends on hormonal factors and cell signals. In a fasting period, the adipocytes provide the energy necessary for the organism to function according to a process known as lipolysis, during which the triglycerides are hydrolyzed into fatty acids and glycerol.

Lipolysis is under the control of a principal enzyme designated triglyceride lipase (TG-lipase). TG-lipase is stimulated directly by cyclic AMP (cAMP). The concentration of cAMP is under the control, on the one hand, of adenylate cyclase whose activation leads to cAMP and, on the other hand, of cAMP phosphodiesterase whose activation is responsible for the breakdown of cAMP. The activity of adenylate cyclase in the adipocytes is regulated by catecholamines and especially adrenalin that is attached to two types of receptors:

the beta adrenergic receptors, which activate adenylate cyclase and, therefore, stimulate lipolysis via the activation of cAMP;

the alpha-adrenergic receptors which inhibit adenylate cyclase and, therefore, also lipolysis.

Therefore, it has been proposed in the prior art to use numerous substances capable of interacting with the alpha and/or beta-adrenergic receptors for stimulating lipolysis. Thus, caffeine is used very much in cosmetics in compositions, especially slimming creams.

Moreover, G proteins discovered by Gilman and Rodbell are known that are involved in the transport system of extracellular hormone towards the cAMP. The G proteins bind the GTP and activate several enzymes including adenylate cyclase which, as indicated previously, promotes the formation of cAMP, which stimulates the TG-lipase and, therefore, lipolysis. Thus, in the prior art it has been proposed to use substances capable of activating the G proteins to increase lipolysis. In the cosmetic field, for example, an extract of Atractyldes Lancea (root) is known; this is a plant of Chinese origin known under the name of Cang Zuh, used in combination with caffeine for the preparation of slimming compositions.

The role is even known of guanylate cyclase, which transforms GTP into cGMP that stimulates the lipases, especially TG-lipase and, therefore, promotes lipolysis.

### SUMMARY OF THE INVENTION

This invention relates to a slimming cosmetic composition including a therapeutically effective amount of plant natriuretic peptide (PNP) as an active agent.

This invention also relates to a method of promoting lipolysis including administering to a mammal a therapeutically effective amount of the composition.

### DETAILED DESCRIPTION

ANP or “atrial natriuretic peptide” is liberated by the cells of the atrium of the heart when the latter is distended by an increase in the volume of blood. Blood transports the ANP to the kidneys where it activates the guanylate cyclase in the cells of the collecting tubes, which promotes formation of cGMP and increases renal secretion of sodium and, as a result, increases the excretion of water which causes a lowering of the volume of the blood.

The presence of receptors for ANP has just now been shown at the adipocyte level, according to the mechanisms described above, which makes it possible to stimulate the guanylate cyclase and, therefore, formation of cGMP that promotes lipolysis via TG-lipase.

The Applicant has now made the most of the role of the ANP to design new compositions promoting lipolysis, comprising ANP as active agent and, more particularly, a plant extract containing ANP useful in the cosmetic field for its slimming properties.

Now, in the cosmetic field, it is quite specifically preferred to use plant extracts. Thus, the invention concerns the use of a plant extract containing ANP as a cosmetic agent stimulating lipolysis for preparing a slimming composition.

In fact, in the plant kingdom, numerous ANP, also designated ANP “like” and also called hereafter PNP for “plant natriuretic peptide”, have been described. They are present in the stems, leaves, petals of plants (David L. Vesely and Amy T. Giordano, Biochemical and Biophysical Research Communications, Aug. 30, 1991, Vol. 179, No. 1, pp. 695-700; J. O. Adeboye et al., 1997, Phytotherapy Research, Vol. No. 11, pp. 454-456, Pulock K.; Mukherjee et al., Phytotherapy Research, 1996, Vol. No. 10, pp. 424-425) like Florida beauty, Buddhist pine, Boston fern, rose, geranium, resurrection plant or club moss, Moses in the cradle, Florid coontie. It is also concerned with the following plants: Vermonia cinerea, Nelumbo nucifera, (sacred lotus), Allium sativum (garlic), Dracena godseffiana, Metasequoia glyptostroboides, Hedera helix (ivy).

Thus, a cosmetic composition according to the invention comprises at least one extract of plants selected from the group comprising: Geranium Robertianu, Vermonia cinerea, Nelumbo nucifera, Allium sativum, Dracena godseffiana, Metasequoia glyptostroboides and Hedera helix.

The compositions according to the invention, therefore, find their application in the treatment of obesity and, more specifically, as a slimming composition in the cosmetic field.

In fact, the body, more specifically of those of women, during the course of time, undergoes unattractive modifications especially of the adipose tissue that is connected to several factors that may be different according to the individuals. Of course, it is related to ageing, but also food or pathological factors. Therefore, in the prior art numerous slimming cosmetic compositions were proposed that were based on plant extracts. More specifically, the slimming cosmetic compositions may be mentioned that are described in the French patent the Applicant published under No. 2 729 856. This patent proposes slimming cosmetic compositions capable of improving and preparing the skin to receive active ingredients by means of soluble organic substances or enzymes, the effects of which are potentiated to promote the removal of lipids and prevent the storage of these lipids.

A slimming cosmetic composition according to the invention is, therefore, characterized in that it comprises PNP as active agent, preferably in the form of a plant extract containing PNP, as a factor stimulating lipolysis and designed to fight against an increase in volume of the adipocytes.

Beside the plant extract containing PNP, the compositions of the invention may comprise other active ingredients useful for inhibiting lipogenesis, or promoting lipolysis, or even for improving, strengthening, lifting the skin surface or moisturizing it. More specifically, a plant extract may be mentioned that is rich is hydroxycitrate, such as a bark extract of the fruit of Garcinia cambodgia. Garcinia cambodgia is a plant originating from the Indies, rich in hydroxycitrate, which is an inhibitor of lipogenesis, therefore, of the storage of fats. Such a bark extract of the fruit of Garcinia cambodgia is marketed under the name of Garcinol, in combination with a biofiltrate of Candida lipolytica. The biofiltrate of Candida lipolytica is obtained by fermentation of this strain on specific medium. It enables stimulation of lipolysis, more specifically, hydrolysis of the triglycerides and, therefore, promotes release of fat from storage.

Besides Garcinol, the slimming compositions according to the invention may comprise caffeine, which has a lipolytic action and reinforces that of Garcinol or even one or more of the following extracts:

an extract of butcher's broom or box holly or even Ruscus, which is rich in inorganic materials such as calcium, potassium, saponosides and sapogenins. The presence of saponosides confers this extract with the properties:

anti-inflammatory, calming and soothing,

effective lightener for protection against redness,

which promotes microcirculation and, therefore, improves the tonicity of the capillaries,

skin decongesting.

one or more derivatives of silicon, such as silanol, which presents the following properties:

moisturizing and enabling regulation of the water concentration,

prevention and regeneration, especially free-radical activity which enables reorganization of the membrane lipids by making the membrane more resistant to the attack of free radicals,

anti-inflammatory, which decreases vasodilation and cancels the action of arachidonic acid.

an extract of the Indian chestnut, which presents the properties:

P vitamins enabling increase of the resistance and decrease of the permeability of the blood capillaries (vasoprotective),

Decongestant and emollients for inflammation because the saponosic derivatives of the Indian chestnut allows the “orange skin” effect to be treated.

An extract of Ginkgo biloba, the dry leaves of which are used in medicine as a vein toner because of their content of flavoids.

A red vine extract. The leaves of red vine are rich in flavonoids and allow the walls of the vessels and the vein tone to be reinforced. Such an extract, therefore, acts on microcirculation.

An extract of red algae, which allows the connective tissue to be protected and to reduce the fragility of the capillaries.

The proportion of the plant extract containing ANP in the compositions according to the invention is between about 0.5 and about 10%, preferably on the order of about 1 to about 5%.

In addition, the slimming cosmetic compositions according to the invention may contain one or more agents capable of providing the composition of the invention with soothing properties or toning properties. Thus, the invention proposes slimming and soothing cosmetic compositions useful for after-sun, after-shaving, special sensitive skins and the like in which the soothing active ingredient is, for example, allantoin, 18-beta-glycyrrhetinic acid, bisabolog, an extract of soothing plants such as linden or camomile.

The invention, therefore, also provides slimming and toning cosmetic compositions in which the toning active ingredient is, for example, ginseng, eleuterococcus, lemon, orange, rathania and the like.

The compositions of the invention may be presented in any form known to the skilled person in the field of cosmetics, such as for cream, milk, lotion, gel, mask and the like without any particular pharmaceutical restriction other than those for application on the skin.

What is claimed is:

1. A slimming cosmetic composition comprising a therapeutically effective amount of plant natriuretic peptide (PNP) as an active agent.

2. The cosmetic composition according to claim 1, wherein the PNP is contained in a plant extract.

3. The cosmetic composition according to claim 2, wherein the plant extract containing PNP is at least one plant extract selected from the group consisting of: Geranium robertianum, Vermonia cinerea, Allium sativum, Dracena godseffiana, Metasequoia glyptostrobodies and mixtures thereof.

4. The cosmetic composition according to claim 1, further comprising at least one agent useful for inhibiting lipogenesis, promoting lipolysis or improving, strengthening or lifting skin surfaces or moisturizing the skin surfaces.

5. The cosmetic composition according to claim 4, comprising an extract from a plant rich in hydroxycitrate.

6. The cosmetic composition according to claim 5, wherein the extract is from bark of Garcinia cambodgia.

7. The cosmetic composition according to claim 1, further comprising caffeine.

8. The cosmetic composition according to claim 1, further comprising at least one of:
an extract of butcher's broom,
one or more derivatives of silicon,
an extract of Indian chestnut,
an extract of Ginkgo biloba,
an extract of red vine, and
an extract of red algae.

9. The cosmetic composition according to claim 1, wherein the proportion of a plant extract containing the PNP is between about 0.5% and about 10%.

10. The cosmetic composition according to claim 1, wherein the proportion of a plant extract containing the PNP is between about 1% and about 5%.

11. The cosmetic composition according to claim 1, wherein the composition is in the form of a cream, a milk, a lotion, a gel or a mask.

12. The cosmetic composition according to claim 1, further comprising an additive selected from the group consisting of allantoin, 18-beta-glycyrrhetinic acid, bisabolog, linden and camomile.

13. A method of promoting lipolysis comprising administering to a mammal a therapeutically effective amount of the composition according to claim 1.
